(19) United States
(12) Patent Application Publication
(10) Pub. No.: US 2004/0110212 A1
(43) Pub. Date: Jun. 10, 2004

(54) Microarrays with visual alignment marks

(76) Inventors: Mark McCormick, Madison, WI (US); Gary Barrett, Liverpool (GB); Roland Green, Madison, WI (US); Jaz Singh, Madison, WI (US)

Correspondence Address: QUARLES & BRADY LLP, FIRSTAR PLAZA, ONE SOUTH PINCKNEY STREET, P.O. BOX 2113 SUITE 600, MADISON, WI, 53701-2113, US

(21) Appl. No.: 10/675,329

(22) Filed: Sep. 30, 2003

Related U.S. Application Data
(60) Provisional application No. 60/415,119, filed on Sep. 30, 2002.

(51) Int. Cl. B05D 3/00; C12Q 1/68; G01N 33/53; C12M 1/34

(52) U.S. Cl. 435/6; 435/7.5; 435/287.2; 427/2.11

(57) ABSTRACT

The present invention provides a method for the in situ synthesis of haptenylated border regions that provide a visual marker for aligning microarrays, and their development by a variety of either chemical or enzymatic methods. The invention also includes microarrays containing the visible borders. The visible borders allow for simple, direct determination of the grid location when pipetting manually, and provide an effective light-scattering marker for determination of grid location by robotic-optical methods.

### CROSS-REFERENCE TO RELATED APPLICATIONS

This application claims the benefit of U.S. Provisional Patent Application Serial No. 60/415,119, filed on Sep. 30, 2002.

### STATEMENT REGARDING FEDERALLY SPONSORED RESEARCH OR DEVELOPMENT

Not applicable.

### BACKGROUND OF THE INVENTION

DNA microarray technology has been applied to many areas such as gene expression and discovery, mutation detection, allelic and evolutionary sequence comparison, genome mapping and more. Unfortunately, most applications fail to tap into the full capacity of microarray technology as many hybridization assays involve far fewer probes than a microarray's full capacity.

The advent of DNA microarray technology makes it possible to build an array of hundreds of thousands of DNA sequences in a very small area, such as the size of a microscopic slide. See, e.g., U.S. Pat. No. 6,375,903 and U.S. Pat. No. 5,143,854, each of which is hereby incorporated by reference in its entirety. The disclosure of Pat. No. 6,375,903 enables the construction of so-called maskless array synthesizer (MAS™) instruments in which light is used to direct synthesis of the DNA sequences, the light direction being performed using a digital micromirror device (DMD). Using an MAS™ instrument, the selection of DNA sequences to be constructed in the microarray is under software control so that individually customized arrays can be built to order. In general, MAS™ based DNA microarray synthesis technology allows for the parallel synthesis of over 786,000 unique oligonucleotides in a very small area of on a standard microscope slide. For many applications, the entirety of the synthesized array is devoted to the evaluation of one biological sample. In these applications, the entire microarray area is enclosed in a small chamber so as to allow for the application of the single sample, thus providing a very efficient means for measuring the concentration of a very large number of target molecules within that one sample. A typical application of this sort is gene expression profiling.

In applications where a smaller number of genes are being studied, or where a reduced set of probes are required for each sample, the microarray can be logically divided into any number of smaller arrays each having the same or different oligonucleotide probes, a concept sometimes referred to as an array of arrays. To use an array of arrays efficiently, multiple samples are hybridized in parallel, in a single experiment, with each sample being hybridized to a given subarray in the array of arrays. This parallel hybridization strategy provides for efficient utilization of the high synthesis capacity of the microarray. In order to load multiple samples onto an array or arrays and avoid sample contamination, some mechanism must be provided to prevent cross contamination of one sample to adjacent samples. Currently, microarrays built for this purpose (e.g., U.S. Pat. No. 5,874,219) use physical barriers (e.g. gaskets, etc.) to separate probe sets for different samples. In these instances, physical barriers have to align with each corresponding probe set to ensure that each well contains the correct probes as any misalignment will lead to inaccurate results.

Proper alignment becomes even more critical when the density of grid elements (i.e. subarrays) is increased to accommodate increased samples. For example, limiting each probe set can increase the number of grid elements to 96, 384 or 1536. Thus, although the arrays can be subdivided in an infinite number of ways, subarrays or grids of the present invention are typically whole number multiples of 96 to coordinate with the source materials which are generally in 96, 384 or 1536 well microtiter plates (standard usage in robotic platforms). Under these conditions, samples must be applied using robotics rather than by manual pipetting. Since the location of the array on the slide can vary (in some instances by more than the diameter of a subarray element), a precise, machine-readable marker becomes essential for accurate, reproducible pipetting of small (1 μl or less) sample volumes.

Regardless of the method used to contain individual samples on the array, determining the precise location of the printed arrays on the slide is critical to correct sample loading. It is therefore desirable to provide a visual marker of the location for each grid element or at least a method for aligning the grid elements to allow for simplified, accurate pipetting of sample.

### BRIEF SUMMARY OF THE INVENTION

The present invention is summarized as a microarray having visible border regions that provide detectable landmarks for determining the position of all grid elements in the microarray. The visible borders allow for simple, direct determination of the location of each grid element (e.g., a subarray containing a probe set) when pipetting manually, and provides an effective machine readable marker for determining grid location using robotic-optical methods.

The visible borders are provided by photopatterning a haptenylated compound onto the microarray in the interstitial regions surrounding the grid elements then rendering visible the haptenylated borders via the use of fluorescent, reflective, refractive or highly contrasting compounds. The compounds may be deposited via one of the methods common to microarray detection and Western blot analysis wherein a binding moiety (e.g. streptavidin) specific to the hapten (e.g. biotin) is supplied in a form coupled to a fluorescent or enzymatic reporter molecule. The reporter molecule may include catalytic antibodies, fluorophore-labeled microparticles, alkaline phosphotases, and horseradish peroxidases. In instances where an enzymatic reporter molecule is used, a supplementary step must be performed where the site-specifically bound enzyme is detected through the addition of a chromogenic substrate. For example, in accordance with the present invention, if alkaline phosphotase is the reporter molecule, its corresponding chromogenic substrate is bromochloro indolyl phosphate/nitro blue tetrazolium (BCIP/NBT).

Accordingly, one aspect of the present invention provides a method for the in situ synthesis of such haptenylated border regions. The border regions are synthesized by photopatterning the haptenylated phosphorarnidite in the region between the grid elements either before, during or at the conclusion of microarray synthesis. In one embodiment, the visible border is synthesized in situ by photopatterning a haptenylated compound, such as biotin phosphoramidite, in the border region and then coupling the biotinylated compound to a secondary compound, such as streptavidin and a reporter molecule, to render the border visible.

A suitable embodiment of the invention is where the entire array is coupled with a NPPOC (2-(2-nitro phenyl)propoxy carbonyl) to provide a patternable first layer. The subarrays are synthesized and the areas to be used for visible borders are reserved (protected from light). The last step in synthesis is the photodeprotection of the areas where visible borders are desired and coupling of biotin-phosphoramidite. This all occurs on the MAS™ instrument so the borders can be placed precisely where desired (i.e., there is flexibility in the placement of the alignment mark within the array). The array is removed from the instrument and deprotected (since the biotin and the array are not functional until deprotected. The biotin borders are then rendered visible or detectable through streptavidin conjugated to a fluorophore, colloidal gold, or other detectable compound. If the reporter is an enzyme, another step is required where precipitable chromogenic substrate described above is added to make the borders visible.

Another aspect of the present invention provides a method for aligning a microarray containing multiple probe sets. The alignment is performed by providing a microarray having visible or machine readable border regions as described above. In manual applications, the location of each grid element is clearly indicated by the visible borders surrounding each grid element. In robotic applications, optical scanning devices may be used to detect high-contrast or light scattering signals resulting from the illuminating compounds. Examples of optical detection devices include the use of a scanning laser diode or the use of image capture and analysis.

### BRIEF DESCRIPTION OF THE SEVERAL VIEWS OF THE DRAWINGS

FIG. 1 shows the chemical structure of 5′-Biotin Phosphoramidite [1-N-4,4′-Dimethoxytrityl)-biotinyl-6-aminohexyl]-2-cyanoethyl-(N,N-diisopropyl)-phosphoramidite (i.e., the molecule used to deliver a hapten onto an array).

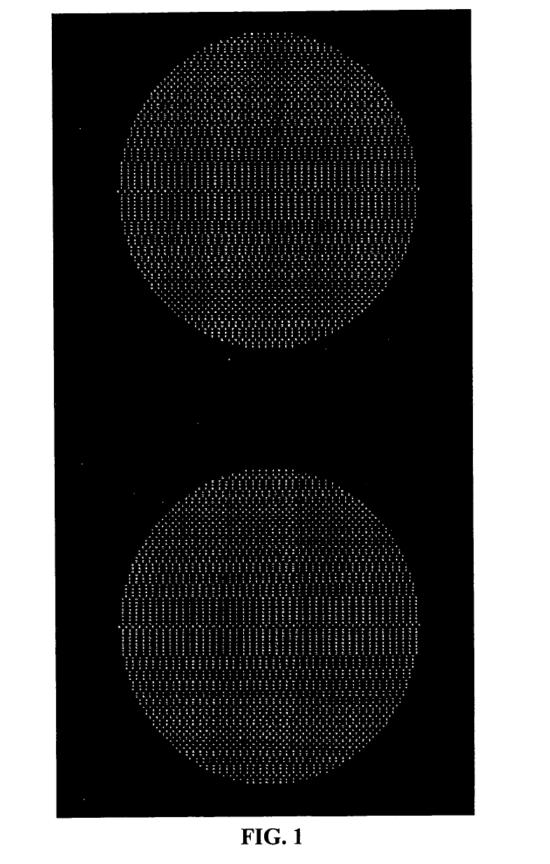

FIG. 2 is a schematic representation of a plurality of subarrays on a microarray showing the visual alignment markers surrounding each subarray.

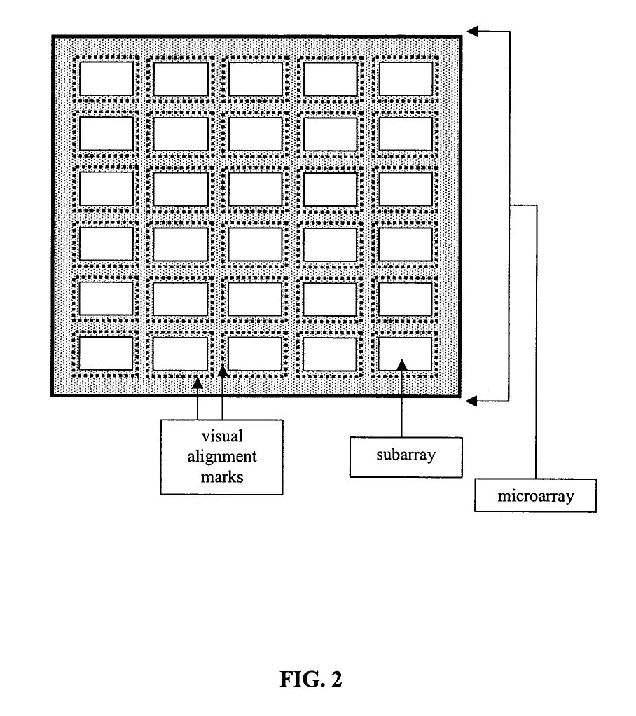

### DETAILED DESCRIPTION OF THE INVENTION

The present invention is a microarray having haptenylated border regions that enable the forming of visual markers for aligning the microarray. The visible markers allow for simple, direct determination of the location of each grid element when pipetting manually, and provides an effective visible marker for determination of grid location by robotic-optical methods. This capability is of particular importance in the use of an array of arrays where different experimental samples are to be delivered to different subarrays in the array. The present invention also provides a method for the in situ synthesis of visible or machine readable (i.e., haptenylated) border regions, and their development using a variety of either chemical or enzymatic methods. The microarray may be a polynucleotide microarray, a polypeptide microarray, or a microarray of other types of molecules.

In practice, the method can be most efficiently accomplished using a slight variation on the normal microarray synthesis. The arrays are synthesized in the conventional way, with nucleotide probe synthesis being limited to subarray or probe areas. A subarray is an area on a microarray slide that contains a set of identical or related probes of interest. A subarray may also contain blank positions (a position available for probes but in which no probes are formed). In making an array of arrays, the subarrays are organized into regions intended to be hybridized to a common experimental sample, here termed a subarray, and entire microarray can contain many such subarrays. The visible markers can be formed in the interstitial areas around each subarray on a microarray. Alternatively, blank positions can be left around each subarray and the blank positions can be incorporated into the border regions to help visualize the borders between subarrays (i.e. visual alignment marks, also referred to herein as visible borders). For the purposes of the present invention, visual alignment markers may be produced with the aid of a variety detection moieties including, among others, streptavidin conjugated to enzymes, catalytic antibodies, colloidal metal suspensions, dyes or fluorophore-labeled microparticles.

The subarrays on a microarray incorporating an array of arrays can optionally by compartmentalized by physical barriers. A compartment so formed is also called a well. Each well would usually be of the same size as or slightly larger than a subarray in all dimensions and is not in fluid communication with other wells during the hybridization process so that a hybridization reaction in one well does not cross-contaminate another. The exact way the subarrays are compartmentalized, or whether they are compartmentalized at all, is not critical for the present invention. For example, a slide pre-patterned with physical barriers can be used to print probes or probes can be printed on a slide first, after which physical barriers are provided on the slide. In the latter case, physical barriers that form each well can be provided individually or as a whole for all wells. For the purpose of the present invention, the shape of each well does not matter and can vary. A suitable configuration of a well will minimize the array area devoted to interstitial uses. A more suitable configuration is a close-face pack of hexagonal or round wells (i.e. in an alternating 3-2-3 or 5-4-5, etc.). Therefore, by using in situ photopatterning to make the visual borders, their alignment with the subarrays is assured irrespective of the shape or configuration of the wells.

In accordance with the present invention, visible borders are constructed around the around subarrays by photopatterning a haptenylated phosphoramidite on the borders of the subarrays. The term photopatterning as used herein is created by the mirrors of the MAS™ instrument. The haptenylated border may be rendered visible by any chemical or enzymatic molecule or combination of molecules that provides a visual output depicting its location. The attachment of the illuminating compound is most easily done by attaching haptens to the array and then attaching fluorescent or light detectable compounds to the haptens. For example, a convenient hapten-based strategy is to attach biotin to the substrate and then use any of the commercially available combinations of streptavidin coupled to any one of the commonly known enzymatic visible reporter molecules, such as alkaline phosphotase or horseradish peroxidase. Streptavidin could be conjugated to colloidal gold nanoparticles, giving rise to visible marker borders from the deposition of gold. Alternatively, DNP (Dintrophenol) to which high affinity monoclonal antibodies are available, or any such similarly used hapten known in the art, may be used instead of biotin. Thus, the visible borders have several elements: haptenylated photopattern, binding moiety (e.g. streptavidin) and reporter molecule (e.g. fluorophore, colloidal gold, enzyme). Where the reporter is an enzyme, a chromogenic substrate may also be required.

The width of the visible border region can be controlled through the chip design, but borders as narrow as 1 mirror width (13 to 171 μm depending on the DMD in use) may be sufficient. To build the visible borders, several alternative strategies are possible. The hapten may be deposited either directly onto the microarray or coupled to the microarray by substituting for a previously deposited compound. In a convenient minor modification of normal MAS™ array synthesis techniques, biotin phosphoramidite is coupled to the array, after array synthesis has concluded, at all desired border areas in the array by photodeprotection of these areas and coupling of the biotinylated phosphoramidite. The appropriate visible marker is then deposited on the array linked to streptavidin. Another convenient technique is to deposit nPC (p-nitrophenyl carbonate) phosphoramidite on the substrate and then to couple an alkyl amine-biotin to nPC via a substitution reaction.

In one aspect of the invention, biotin is coupled to the array during synthesis as a phosphoramidite. In those locations where a visible border is desired, that area is photodeprotected using the MAS™ instrument (as part of the synthesis) and biotin-phosphoramidite is coupled to the phototyprotected area. The array is deprotected (i.e., the instrument is off) and each subarray is surrounded by biotin (the hapten) borders. The borders are made visible by binding streptavidin conjugated to something that can provide a detectable endpoint such as SA-Cy3, SA-AP, SA-HRP. If an enzymatic reporter molecule is used, an additional development step is required to deposit chromogenic, precipitable substrate wherever the enzyme is located.

In a more suitable aspect of the invention, the use of biotin as an affinity handle allows for highly specific docking of a variety of detection moieties using biotin-streptavidin interactions, including enzymes or catalytic antibodies, colloidal metal suspensions, dye or fluorophore-labeled microparticles. Specific detection methods may include the use of enzymatic reporter molecules such as alkaline phosphotase or horseradish peroxidase in combination with precipitable, chromagenic substrates such as bromochloro indolyl phosphate/nitro blue tetrazolium (BCIP/NBT). Other examples applicable to the present invention include the docking of streptavidin conjugated colloidal gold nanoparticles. The use of colloidal metal suspension provides several advantages including the development of high contrast light scattering borders and the option to enhance the signal through the use of metal deposition chemistry (for example silver enhancement of colloidal gold).

For the manual application of samples to low-density sub array grids, the location of grid elements is clearly indicated by the visible borders in the interstitial regions between each grid element. For robotic applications, the method of detection also relies on the chemical or enzymatic development of high-contrast or light scattering signal on the borders to be visualized. Also the detection step involves the visualization of the borders through scanning optics. Examples of optical detection include the use of a scanning laser diode or the use of image capture and analysis. These and other methods for grid registration rely on the generation of high contrast between the labeled border region and the surrounding, unlabeled region.

The present invention is most useful for an application in which a hybridization assay is used to analyze a large number of samples. It is understood, however, that examples and embodiments of the present invention set forth above are illustrative and not intended to confine the invention. The invention embraces all modified forms of the examples and embodiments as come within the scope of the following claims.

We claim:

1. A microarray comprising a plurality of subarrays wherein at least one subarray contains a set of nucleic acid probes of interest, and wherein at least one subarray is surrounded by an interstitial region; wherein the interstitial region comprises at least one visible or machine readable alignment marked conformed by photopatterning a group-bearing phosphoramidite onto the interstitial region of a microarray.

2. The microarray of claim 1 wherein the alignment mark comprises a hapten and an illuminating compound.

3. The microarray of claim 2 wherein the hapten is a biotin or DNP.

4. The microarray of claim 2 wherein the illuminating compound is streptavidin-conjugated to a reporter molecule.

5. The microarray of claim 4 wherein the reporter molecule is selected from the group consisting of a catalytic antibody, colloidal metal suspension, dye, fluorophore-labeled microparticles, alkaline phosphotase, and horseradish peroxidase.

6. The microarray of claim 1 wherein the alignment mark is flexibly deployable within the array and can be placed with great precision immediately adjacent to and surrounding the subarray.

7. A method for making a microarray having a plurality of subarrays surrounded by a visible or machine readable alignment mark in an interstitial region of the microarray, the method comprising the steps of:
a) selecting at least one probe set comprising probes of interest;
b) building the probe sets on a microarray slide to provide a plurality of subarrays; and
c) depositing between subarrays a hapten and an illuminating compound to form the alignment mark between the subarrays on the microarray.

8. The method of claim 7 wherein the hapten comprises is a biotin or DNP.

9. The method of claim 7 wherein the illuminating compound is streptavidin conjugated to a reporter molecule.

10. The method of claim 9 wherein the streptavidin is bound to a reporter molecule selected from the group consisting of a catalytic antibody, colloidal metal suspension, dye, fluorophore-labeled microparticles, alkaline phosphotase, and horseradish peroxidase.

11. The method of claim 6 wherein the hapten is deposited by photopatterning a group-bearing phosphoramidite onto the interstitial region of the microarray.

12. The method of claim 11 wherein the phosphoramidite is NPPOC.

13. The method of claim 7 wherein the alignment mark is flexibly deployable within the array and can be placed with great precision immediately adjacent to and surrounding the subarray.

14. A method for aligning microarrays, the method comprising the steps of:
a) providing the microarray of claim 1;
b) exposing the microarray to an optical detection device to detect the visible or machine readable alignment mark on the interstitial region surrounding the subarrays; and
c) aligning the microarray according to the location of the visible or machine readable alignment mark so as to accurately deposit samples into the subarrays of a microarray.

15. The method of claim 14, wherein the optical detection device is either a scanning laser diode or an image capture and analysis device.

16. The method of claim 14, wherein the samples are deposited into the subarrays using robotics.
